(19) United States
(12) Patent Application Publication
(10) Pub. No.: US 2009/0326342 A1
(43) Pub. Date: Dec. 31, 2009

(54) METHOD, ARRANGEMENT AND SENSOR FOR NON-INVASIVELY MONITORING BLOOD VOLUME OF A SUBJECT

(75) Inventor: Matti Huiku, Espoo (FI)

Correspondence Address: Andrus, Sceales, Starke & Sawall, LLP, 100 East Wisconsin Avenue, Suite 1100, Milwaukee, WI, 53202-4178, US

(73) Assignee: THE GENERAL ELECTRIC COMPANY, Schenectady, NY (US)

(21) Appl. No.: 12/163,305

(22) Filed: Jun. 27, 2008

(51) Int. Cl. A61B 5/1455

(52) U.S. Cl. 600/322

(57) ABSTRACT

A method, arrangement and sensor for monitoring blood status of a subject are disclosed. In-vivo measurement signals indicative of absorption caused by blood are acquired at a plurality of measurement wavelengths. Based on the in-vivo measurement signals, successive values are determined for a hemoglobin parameter indicative of the concentration of hemoglobin in the blood of the subject and the blood volume status of the subject is monitored based on the successive values. The monitoring may involve determining the absolute value of the blood volume or relative changes in the blood volume. In one embodiment, the absolute value of the blood volume is indicated continuously together with hemoglobin concentration and composition.

### BACKGROUND OF THE INVENTION

This disclosure relates generally to monitoring of blood volume in a subject. More specifically, this disclosure relates to monitoring of blood volume with the help of hemoglobin concentration in blood, which may be determined non-invasively and continuously, without blood sampling, by a pulse oximeter based measurement at multiple wavelengths.

Monitoring of blood volume requires that the concentration of a blood substance is measured. Traditionally, the blood volume of a subject has been estimated through an in-vitro analysis of one or more blood samples taken from the subject, by determining the hemoglobin dilution in the samples, i.e., hemoglobin is used as the blood substance whose concentration change is determined before and after diluting the blood with a known amount of fluid. The hemoglobin concentration can be measured by devices known as co-oximeters that determine the concentration by measuring spectral light transmission/absorption through a hemolysed blood sample at several wavelengths, typically between 500 and 650 nm.

A major drawback related to co-oximeters is that the measurements are invasive, i.e. require a blood sample to be taken from the patient. Furthermore, the co-oximeters are rather expensive laboratory devices and require frequent service and maintenance.

In order to obviate the continuous blood sampling, it has also been suggested to use pulse oximeter technology for measuring the concentration of a tracer substance in blood. In a method like this, the blood hemoglobin concentration is determined first by taking a blood sample. A known amount of a tracer substance, such as indocyanine green, is then injected into the subject and the concentration of this substance in the blood is tracked using pulse oximeter technology. The determination of the tracer substance concentration requires that the hemoglobin concentration determined previously is used as reference. Based on the tracer concentration, blood volume may be determined.

Although the use of a tracer substance allows blood volume to be monitored without successive blood sampling, known methods do not suit well for long-term or continuous monitoring of the blood volume. This is partly because the said methods are discrete in the sense that the measurements must be carried out before the tracer substance is eliminated from the body. Long-term tracking of blood volume thus requires that the tracer substance is retained in the blood for longer periods, which may be achieved either by repeating the injection after the previous bolus of tracer substance is removed from the plasma or by using a tracer substance that retains in the plasma for longer periods. However, long-term use of tracer substances is not desirable, due to the possible side effects that the tracer substances may have.

Thus, the drawback of the current technology is that it does not allow long-term blood volume monitoring without long-term retainment of a tracer substance in blood.

### BRIEF DESCRIPTION OF THE INVENTION

The above-mentioned problems are addressed herein which will be comprehended from the following specification.

In an embodiment, a method for monitoring the blood volume status of a subject comprises acquiring in-vivo measurement signals at a plurality of measurement wavelengths, the in-vivo measurement signals being indicative of absorption caused by the blood of the subject. The method further comprises determining, based on the in-vivo measurement signals, a hemoglobin measure indicative of concentration of hemoglobin in the blood of the subject, wherein the determining includes determining successive values of the hemoglobin measure, and monitoring, based on the successive values of the hemoglobin measure, the blood volume of the subject.

In another embodiment, an arrangement for monitoring the blood volume status of a subject comprises a signal reception unit configured to receive in-vivo measurement signals corresponding to a plurality of measurement wavelengths, the in-vivo measurement signals being indicative of absorption caused by the blood of the subject, a hemoglobin determination unit configured to determine, based on the in-vivo measurement signals, a hemoglobin measure indicative of the concentration of hemoglobin in the blood of the subject, wherein the hemoglobin determination unit is configured to determine successive values of the hemoglobin measure, and a monitoring unit configured to monitor, based on the successive values of the hemoglobin measure, the blood volume of the subject.

In yet another embodiment, a sensor attachable to a subject for determining the blood volume status of the subject comprises an emitter unit configured to emit radiation through the tissue of the subject at a plurality of measurement wavelengths and a detector unit configured to receive the radiation and to produce in-vivo measurement signals corresponding to the plurality of measurement wavelengths, wherein the in-vivo measurement signals are indicative of absorption caused by the blood of the subject. The sensor further comprises a hemoglobin determination unit configured to determine, based on the in-vivo measurement signals, a hemoglobin measure indicative of the concentration of hemoglobin in the blood of the subject, wherein the hemoglobin determination unit is configured to determine successive values of the hemoglobin measure, and an interface unit configured to send the successive values of the hemoglobin measure to an external unit configured to monitor the blood volume of the subject based on the successive values.

In a further embodiment, an apparatus for monitoring the blood volume status of a subject comprises a reception unit configured to receive successive values of a hemoglobin measure indicative of hemoglobin concentration in the blood of the subject and a monitoring unit configured to monitor, based on the successive values of the hemoglobin measure, the blood volume of the subject.

In a still further embodiment, a computer program product for monitoring the blood volume status of a subject comprises a first program product portion configured to receive in-vivo measurement signals corresponding to a plurality of measurement wavelengths, the in-vivo measurement signals being indicative of absorption caused by the blood of the subject, a second program product portion configured to determine, based on the in-vivo measurement signals, successive values of a hemoglobin measure indicative of haemoglobin concentration in the blood of the subject, and a third program product portion configured to indicate, based on the successive values, blood volume status of the subject.

Various other features, objects, and advantages of the invention will be made apparent to those skilled in the art from the following detailed description and accompanying drawings.

### BRIEF DESCRIPTION OF THE DRAWINGS

FIG. 1 is a block diagram illustrating one embodiment of a pulse oximeter monitoring blood volume;

FIG. 2 is a flow diagram illustrating an embodiment in which relative changes in the blood volume are monitored;

FIG. 3 is a flow diagram illustrating another embodiment in which the blood volume status is monitored by continuously determining the absolute value of the blood volume of the subject;

FIG. 4 illustrates a simple model based on the Lambert-Beer theory of pulse oximetry;

FIG. 5 illustrates one embodiment for the determination of hemoglobin concentration in the methods of FIGS. 2 and 3;

FIG. 6 illustrates the actual in-vivo and Lambert-Beer model based light transmissions in tissue;

FIG. 7a to 7f illustrate examples of transformations defining the relationship between the in-vivo modulation ratio N(in-vivo) and the Lambert-Beer modulation ratio N(L-B);

FIG. 8a to 8f illustrate the in-vivo measured and theoretical Lambert-Beer modulation ratios as a function of SpO₂;

FIG. 9 illustrates the path length multiplier PLM as a function of an expansion parameter Σₐ/Σ′ₛ;

FIG. 10 is a flow diagram illustrating a further embodiment for the determination of hemoglobin concentration in the methods of FIGS. 2 and 3;

FIG. 11 is flow diagram illustrating an embodiment of the blood volume determination; and

FIG. 12 illustrates one embodiment of the apparatus of the invention.

### DETAILED DESCRIPTION OF THE INVENTION

A pulse oximeter normally comprises a computerized measuring unit and a probe attached to the patient, typically to a finger or ear lobe. The probe includes a light source for sending an optical signal through the tissue and a photo detector for receiving the signal transmitted through or reflected from the tissue. On the basis of the transmitted and received signals, light absorption by the tissue may be determined. During each cardiac cycle, light absorption by the tissue varies cyclically. During the diastolic phase, absorption is caused by venous blood, non-pulsating arterial blood, cells and fluids in tissue, bone, and pigments, whereas during the systolic phase there is an increase in absorption, which is caused by the inflow of arterial blood into the tissue part on which the sensor is attached. Pulse oximeters focus the measurement on this pulsating arterial blood portion by determining the difference between the peak absorption during the systolic phase and the background absorption during the diastolic phase. Pulse oximetry is thus based on the assumption that the pulsatile component of the absorption is due to arterial blood only.

In order to distinguish between two species of hemoglobin, oxyhemoglobin (HbO₂) and deoxyhemoglobin (RHb), absorption must be measured at two different wavelengths, i.e. the probe of a traditional pulse oximeter includes two different light emitting diodes (LEDs) or lasers. The wavelength values widely used are 660 nm (red) and 940 nm (infrared), since the said two species of hemoglobin have substantially different absorption at these wavelengths. Each LED is illuminated in turn at a frequency which is typically several hundred Hz.

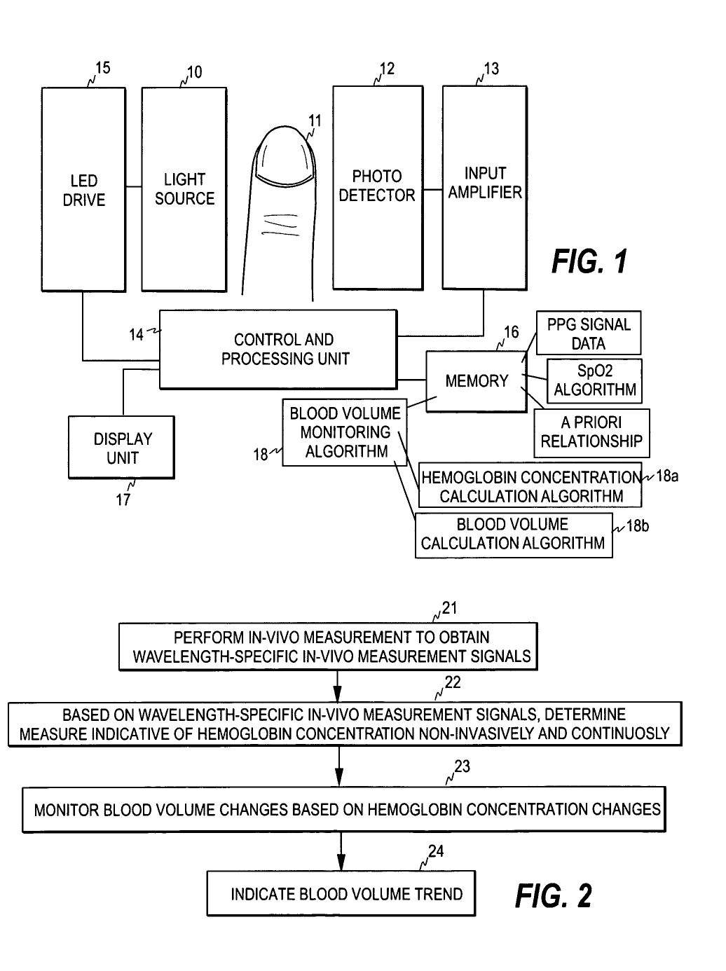

FIG. 1 is a block diagram of one embodiment of a pulse oximeter for monitoring blood volume. Light transmitted from a light source 10 including a plurality of LEDs or lasers passes into patient tissue, such as a finger 11. As discussed below, the number of wavelengths used in the pulse oximeter may vary. However, at least two LEDs (wavelengths) are required for oxygen saturation measurement.

The light propagated through or reflected from the tissue is received by a photodetector 12, which converts the optical signal received at each wavelength into an electrical signal pulse train and feeds it to an input amplifier 13. The amplified signal is then supplied to a control and processing unit 14, which converts the signals into digitized format for each wavelength channel. The digitized signal data is then utilized by an SpO₂ algorithm. The control and processing unit executes the algorithm and drives a display 17 to present the results on the screen of the display. The SpO₂ algorithm may be stored in a memory 16 of the control and processing unit.

The control and processing unit further controls a source drive 15 to alternately activate the LEDs. As mentioned above, each LED is typically illuminated several hundred times per second. The digitized photoplethysmographic (PPG) signal data at each wavelength may also be stored in the said memory before being supplied to the SpO₂ algorithm.

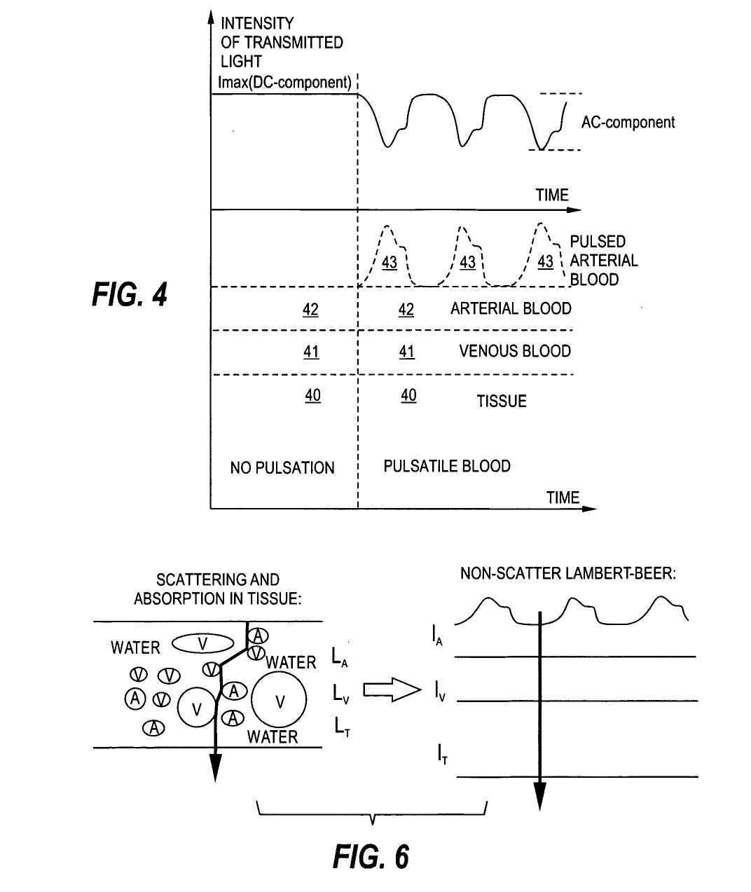

With each LED is illuminated at such a high rate as compared to the pulse rate of the patient, the control and processing unit obtains a high number of samples at each wavelength for each cardiac cycle of the patient. The value of these samples varies according to the cardiac cycle of the patient, the variation being caused by the arterial blood, as is shown below in FIG. 4.

In order for variations in extrinsic factors, such as the brightness of the LEDs, sensitivity of the detector, or thickness of the finger, to have no effect on the measurement, each signal received is normalized by extracting the AC component oscillating at the cardiac rhythm of the patient, and then dividing the AC component by the DC component of the light transmission or reflection. The signal thus obtained is independent of the above-mentioned extrinsic factors.

A conventional pulse oximeter of the above type is upgraded with a mechanism for monitoring the blood volume status of a subject, i.e., the absolute blood volume and/or relative changes therein. For this purpose, a blood volume monitoring algorithm 18 may be stored in the memory of the pulse oximeter. The algorithm may be divided into two logical modules; a first module 18a for the determination of the concentration of hemoglobin and a second module 18b for the determination of a parameter indicative of the blood volume or changes therein. The control unit executes the algorithm which may utilize the same digitized signal data as the SpO₂ algorithm or the results derived in the SpO₂ algorithm. As discussed below, as compared to a standard two-wavelength pulse oximeter, the pulse oximeter intended for the determination of blood volume is further provided with extra wavelengths and may further be provided with a dedicated sensor, for example. However, the operation of the blood volume monitoring algorithm is discussed first with reference to embodiments in which hemoglobin is used as a tracer substance and the concentration of hemoglobin is determined by requiring that the effect of the in-vivo tissue on the in-vivo signals is consistent for all wavelengths at which the in-vivo measurement is performed.

FIG. 2 illustrates an embodiment for monitoring relative changes in the blood volume of a subject. In-vivo measurement signals are measured from in-vivo tissue at different wavelengths of a pulse oximeter (step 21). In-vivo measurement signals here refer to signals obtained from a living tissue. Based on the wavelength-specific in-vivo measurement signals obtained, a hemoglobin measure indicative of total hemoglobin concentration in the blood of the subject is determined step (22). It is assumed henceforward that the hemoglobin measure corresponds to the actual hemoglobin concentration (g/dl or equivalent units) or the volume fraction of the red cells containing hemoglobin, i.e., hematocrit, that directly indicates the hemoglobin concentration in the whole blood, as well. The determination of the hemoglobin measure is carried out non-invasively and substantially continuously, whereby successive values are obtained for the hemoglobin concentration. As discussed below, the determination of the hemoglobin concentration may be based on a theoretical relationship indicative of the effect of tissue on in-vivo measurement signals at the wavelengths of the apparatus, since such an embodiment brings along significant advantages, such as easy determination of the absolute blood volume and hemoglobin composition (i.e. the concentration of different hemoglobin species). However, in order to monitor relative changes in blood volume, any pulse oximeter based method that enables continuous tracking of hemoglobin concentration may be used. Based on the relative changes in the hemoglobin concentration, relative changes in the blood volume are determined (step 23) and blood volume trend is indicated to the end-user (step 24). The determination of the relative blood volume changes is based on the known hemodilution principle according to which the blood volume at time instant t can be determined as follows:

$${V}_{blood} ( t ) = {V}_{blood} ( {t}_{0} ) \times \frac{THb ( {t}_{0} )}{THb ( t )} ,$$

where V_{blood}(t₀) represents blood volume at time t=t₀ and THb(t) and THb(t₀) represent total hemoglobin concentrations, i.e. the hemoglobin measure, at time instants t and t₀, respectively. As the hemoglobin measure THb(t) is tracked continuously, relative changes in the blood volume may be tracked continuously without a need to determine the absolute value of the blood volume. However, the said absolute value is determined in the embodiments discussed below.

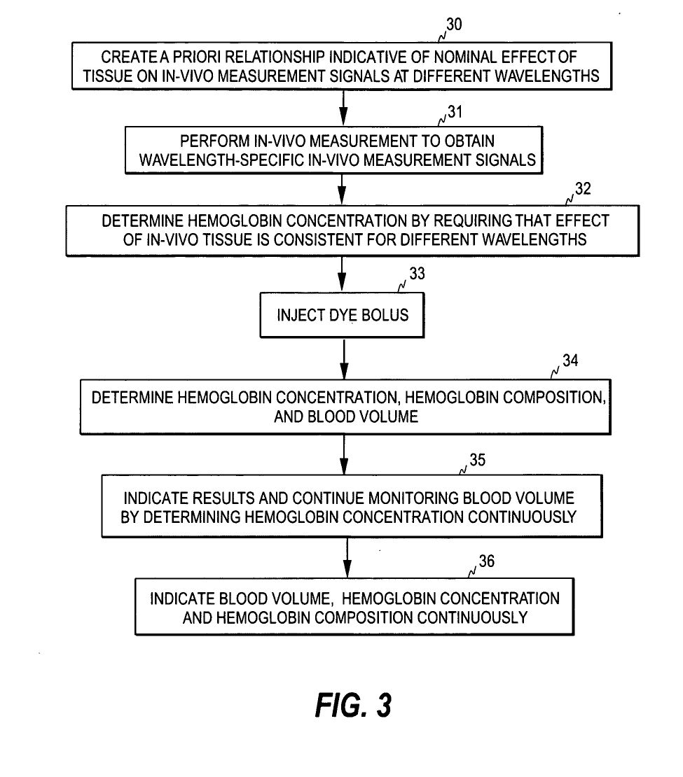

FIG. 3 illustrates another embodiment, in which (total) hemoglobin concentration, hemoglobin composition, and the absolute value of blood volume may be monitored continuously. In this embodiment, an a priori relationship is formed, which is indicative of the (nominal) effect of the tissue on in-vivo measurement signals at the wavelengths of the apparatus (step 30). As is discussed below, the a priori relationship may be formed empirically but the use of a tissue model is beneficial for the determination of the a priori relationship, since the model based approach requires considerably less work and provides a better solution in terms of medical ethics.

The in-vivo measurement signals are then measured from in-vivo tissue at different wavelengths (step 31). The concentration of a substance in the blood, such as hemoglobin, may be determined based on the a priori relationship by requiring that the effect of the in-vivo tissue on the in-vivo signals remains consistent for all wavelengths at which the in-vivo measurement is performed (step 32). Consistency may be found based on the a priori relationship.

When the monitoring of blood volume is started, an initial volume calibration is carried out by injecting a dye bolus to the subject (step 33). After the dye bolus is distributed evenly into the blood circulation of the subject, hemoglobin concentration, hemoglobin composition, and blood volume may be determined at current time instant (step 34). The determination of the hemoglobin concentration in step 34 is carried out similarly as in steps 31 and 32, and the blood volume is determined based on dye concentration which is also determined. The hemoglobin composition is obtained in connection with the determination of the dye concentration. However, as discussed below, hemoglobin composition may be determined with or without dye substance, i.e., in the disclosed mechanism the dye substance serves for the volume calibration only, while hemoglobin serves as a dye substance that is confined naturally in blood and forms the basis of a continuous volume measurement.

The results obtained in step 34 are then indicated to the end-user and the monitoring of blood volume is continued by continuously determining the hemoglobin concentration (step 35). The continued determination of the hemoglobin concentration is carried out as in steps 31 and 32.

Thus, in the embodiment of FIG. 3 one injection of a dye substance is given to the subject, after which the blood volume of the subject may be tracked continuously by continuously determining the hemoglobin concentration. Long-term blood volume monitoring is therefore possible without blood sampling and without long-term use of foreign tracer substances. Hemoglobin composition may be determined simultaneously with hemoglobin concentration, whereby blood volume, hemoglobin concentration, and hemoglobin composition may be indicated to the end-user in the continuous state, i.e. after the dye bolus and after the elimination of the dye substance from the body (step 36). The dye bolus may be given at any appropriate time, such as before a medical procedure requiring the monitoring of blood volume.

Below, the embodiment of FIG. 3 is disclosed in more detail by first discussing the determination of hemoglobin concentration carried out in step 32.

FIG. 4 illustrates the Lambert-Beer tissue model and how the intensity of light transmitted through a finger, for example, varies according to blood pulsation. The determination of the hemoglobin is based on a relationship between the in-vivo measured PPG signals and wavelength-specific values of a predetermined parameter indicative of the wavelength-dependent effect of the in-vivo tissue on the measured signal and thus also on the consistency of the effect at different wavelengths. The relationship defines how values may be derived for the predetermined parameter from the in-vivo signals.

In-vivo based values of the predetermined parameter are examined to find out when consistency occurs for the wavelengths at which the in-vivo measurement is made. One tissue model that may be utilized in the model based approach is a model that is based on the known Lambert-Beer theory. FIG. 4 illustrates a simple model for the Lambert-Beer theory of pulse oximetry. The theory is based on a multilayer model in which light absorption is caused by different tissue compartments or layers stacked on each other. As illustrated in the figure, the tissue compartments include the actual tissue layer 40, layers of venous and arterial blood, 41 and 42, and the layer of pulsed arterial blood 43. The model assumes that the layers do not interact with each other and that each layer obeys the ideal Lamber-Beer model, in which light scattering is omitted. The pulsed signal (AC) measured by a pulse oximeter in the Lambert-Beer model is thus the signal that is left when the absorption caused by each layer is deducted from the input light signal. The total absorption may thus be regarded as the total absorption caused by the actual tissue, venous blood, arterial blood, and pulsed arterial blood.

In order to determine the concentration of a blood substance, an in-vivo tissue model may thus be used, which includes a tissue parameter representing the concentration of a desired blood substance, such as hemoglobin. The in-vivo tissue model is such that it adds interactions between the ideal Lambert-Beer layers, i.e. in the model the in-vivo signals are affected by the absorbing and scattering tissue components specified in the Lambert-Beer tissue model for layers 40-43. The three layers 40-42 beneath the pulsed arterial blood are in this context termed the background, since they form a “background” for the pulsatile component of the absorption (i.e. for the AC-component of the measurement signal).

As discussed above, in one embodiment the a priori relationship created in step 30 of FIG. 3 is based on the above-mentioned in-vivo tissue model obtained by adding interactions to a known model, such as the Lambert-Beer model. The tissue model obtained typically includes a number of parameters, one of the parameters being the above-mentioned tissue parameter, i.e. a parameter which is indicative of the concentration of a desired blood substance, such as hemoglobin. The a priori relationship may be created with nominal tissue parameter values and the relationship may describe the effect of the tissue on a predetermined parameter derivable from the in-vivo signals, wherein the parameter is such that the effect, which is wavelength-dependent, may be seen in it. As discussed below, the predetermined parameter derivable from the in-vivo signals may be such that background color and/or color density is/are reflected in the value of the parameter.

Consistency is detected based on the predetermined parameter and the a priori relationship. However, the criterion indicating the occurrence of consistency depends on the predetermined parameter utilized. In one embodiment, a theoretical value for the predetermined parameter is determined. This theoretical value may be calculated using an ideal tissue, such as only the pulsating arterial blood in the Lambert-Beer model. An in-vivo measurement is then performed (steps 21 and 31) and based on the measurement at least one in-vivo based value is determined for the predetermined parameter. However, typically several wavelength-specific in-vivo based values are determined. The a priori relationship is then altered by adjusting the value of the tissue parameter so that it yields the best possible agreement between the in-vivo based values and the theoretical values of the predetermined parameter, i.e. the value of the tissue parameter is searched for, for which the in-vivo based values and the theoretical equivalent(s) correspond to each other. This value of the tissue parameter is regarded as the actual concentration of the blood substance.

The above-described a priori relationship may be created in the manufacturing phase of the apparatus and stored in the memory of the apparatus. In connection with an in-vivo measurement, the apparatus may then determine, based on the relationship and in-vivo measurement signals, a set of wavelength-specific values for the predetermined parameter. The consistency of the wavelength-specific values is checked based on the a priori relationship and if consistency is not found directly, the a priori relationship is adjusted so that the set of wavelength-specific values indicate consistency. The value of the tissue parameter that yields the consistency determines the concentration.

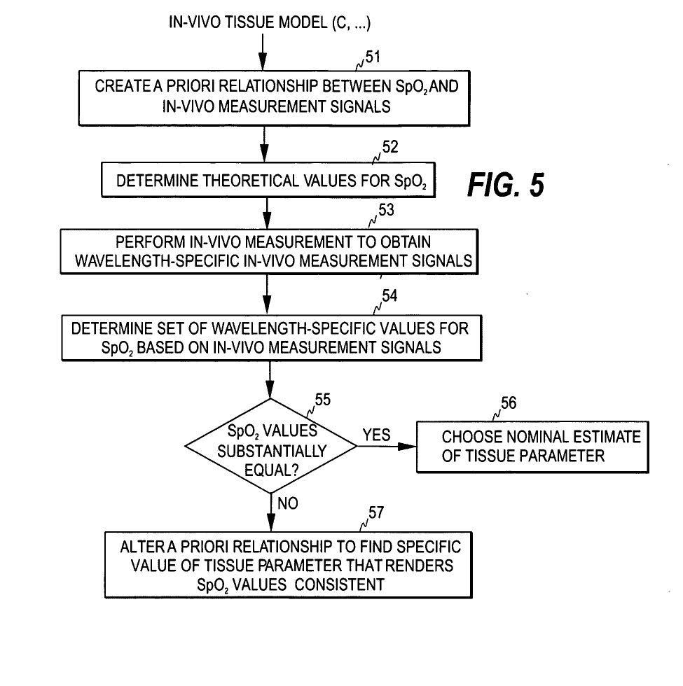

FIG. 5 illustrates an embodiment, in which the predetermined parameter represents arterial oxygen saturation, SpO₂. Conventional oximeters calculate SpO₂ from signals measured at two wavelengths, typically, as mentioned before, at 660 nm and 940 nm. However, the oxygen saturation can as well be determined from any other two wavelengths. When more than two wavelengths are employed in a pulse oximeter, the rule of consistency is that the same saturation percentage must be obtained from any wavelength pair. For instance, if there are three wavelengths, say 650 nm, 760 nm and 880 nm, the first SpO₂ value can be determined from 650 nm and 760 nm, the second value from 650 nm and 880 nm, and a third estimate for SpO₂ from 760 nm and 880 nm. The oxygen saturation SpO₂, i.e. the oxyhemoglobin fraction in percentage, must be the same for all wavelength pairs. In this case an a priori relationship is thus formed between the SpO₂ and the in-vivo signals measured at the wavelengths of the apparatus (step 51). The a priori relationship can be such that it maps, at each wavelength pair, the ratio of measured AC/DC-signals to an SpO₂ value. The nominal relationships between the signal ratios and SpO₂, i.e. the mapping functions, may be stored in the memory of the apparatus (step 52).

In-vivo measurements are then made using several wavelength pairs (step 53) and an in-vivo based set of SpO₂ values is determined based on the in-vivo measurement signals and the relationships (step 54). Since SpO₂ values may change through time, consistency is achieved for the different wavelengths if it is detected that the in-vivo based SpO₂ values obtained in the measurement are essentially the same. The values are compared with each other at step 55. However, if it is detected at step 55 that the SpO₂ values deviate substantially from each other, inconsistency is detected. The concentration value is then sought for at step 57, which yields a minimum difference between the in-vivo based SpO₂ values.

The concentration value obtained corresponds to a situation in which the effect of the in-vivo tissue on the measured in-vivo signals is consistent for the wavelengths at which the SpO₂ values were measured. In case of SpO₂ being the predetermined parameter, the consistency requirement means that the arterial blood color seen against a varying background color and color density must be the same and independent of the background properties. Arterial blood thus serves as a color marker, which must be detected consistently at all wavelengths regardless of the background properties. In analogous simple terms, to an eye the color of an object seems to depend on the background against which the object is seen. However, although the object looks differently, the object's true color is the same. In this case the object is the arterial blood, the true color corresponds to the arterial saturation, SaO2, to which all other tissue components form the background.

In summary, the above-described determination of hemoglobin concentration is based on a general principle of using arterial hemoglobin (pulsating hemoglobin) as a marker, which must be seen the same independent of the background tissue. By requiring that the true color must be invariant, the properties of the background can actually be determined. The concentration of total hemoglobin or glucose or any other blood substance in the background can thus be determined using this principle.

Next, the SpO₂-based embodiment shown in FIG. 5 is discussed in more detail with reference to FIGS. 6 to 9.

SpO₂ Within the Lambert-Beer Model

Within the Lambert-Beer model, the transmitted light through the tissue layers can be expressed mathematically as follows: Iₒᵤₜ=Iᵢₙ×exp(−Σ(cᵢ×εᵢ×lᵢ), (1),
where Iᵢₙ is the light intensity input and Iₒᵤₜ is the light intensity output, cᵢ is the concentration of the color substance in layer i, εᵢ is the extinction coefficient of the color substance in layer i, and lᵢ is the thickness of layer i. The basic oximeter equation can be obtained by differentiating the transmitted intensity with time and remembering that the only time variant absorption is due the arterial blood, which results in:

AC/DC(within L-B)=ΔI/I=−cₐ×εₐ×lₐ (2),
where AC and DC refer to the AC and DC components of light transmission (cf. FIG. 4), ΔI refers to the pulsatile transmitted light intensity, l refers to the total transmitted light intensity, subscript a refers to arterial blood, Ea refers to the extinction coefficient of the arterial blood, cₐ to the concentration of the substance in blood, and lₐ represents the thickness of the pulsating, time variant blood layer (layer 43 in FIG. 4).

In pulse oximeters, the light transmission measurement is performed at two wavelengths, red and infrared, respectively. The ratio of the AC/DC ratios at these wavelengths is in this context termed modulation ratio and denoted with Nₖₗ where the subscripts k and l refer to the wavelengths. The AC/DC ratio at wavelength i is denoted with dAi. Consequently, Nₖₗ=dAₖ/dAₗ. By assuming a Lambert-Beer model for the absorption in arterial blood and that there are only two hemoglobin species, oxyhemoglobin and deoxyhemoglobin, in blood with respective fractions SpO₂/100 and (100−SpO₂)/100, an ideal L-B relationship is obtained:

$${SpO}_{2 kl} = \frac{{ɛ}_{kHb} - {N}_{kl} * {ɛ}_{lHb}}{{N}_{kl} * ( {ɛ}_{iHbO 2} - {ɛ}_{IHb} ) - ( {ɛ}_{kHbO 2} - {ɛ}_{kHb} )} , \qquad ( 3 )$$

where the wavelengths are denoted by k and l, Nₖₗ is the above modulation ratio for the wavelengths k and l, ε is the extinction coefficient, and HbO2 and Hb refer, respectively, to oxyhemoglobin and deoxyhemoglobin.

The Concept of a Path Length Multiplier

FIG. 6 illustrates the principles of establishing the relationship between the measured in-vivo light signal and the non-scatter light signal within the Lambert-Beer tissue model. Due to scattering, the actual light path through the tissue is longer than in the Lambert-Beer model. The relationship between the in-vivo measured signals and the corresponding signals within the model can be constructed by calculating, at each wavelength, a path length multiplier (PLM), which describes how much longer the actual light path through a particular tissue layer is in comparison to the ideal straight line. PLM is thus a measure for the effect of light scattering in tissue: the larger the scattering relative to absorption, the longer the actual light path length through the tissue. With constant scattering, the light path shortens as absorption increases. The calculation of PLM will be described in more detail below.

With the help of the PLM concept, the actual pulse oximeter signal can be expressed as follows:

AC/DC (in-vivo)=ΔI/I=−cₐ×εₐ×Lₐ, (4),
where La is the real path length through the pulsating arterial blood. Using the PLM, it may then be written:

Lₐ=PLM(λ, Σₐ, Σₛ)×lₐ, (5),
i.e., Lₐ is a function of wavelength, total absorption (Σₐ), and scattering (Σₛ) of the tissue (by all tissue layers/components).

Alternatively, the above equation may be expressed by the equation:

AC/DC(in-vivo)=ΔI/I=−cₐ×PLM×εₐ×lₐ, (6)
which defines an in-vivo extinction coefficient ε′ₐ as follows:

ε′ₐ=PLM(λ,Σₐ,Σₛ)×εₐ. (7)

The path length multiplier PLM can thus be thought to alter either the extinction coefficients or the path lengths within the Lambert-Beer theory.

To sum up, PLM is a function of wavelength, scattering and absorption, i.e. color and color density of the absorbing tissue layers of the background and arterial blood.

The Transformation Between the In-Vivo Signals and the Fictitious Lambert-Beer Model Signals

Next, the path length multiplication concept is utilized to mathematically establish a relationship between the in-vivo measured signals and the fictitious signals in the Lambert-Beer tissue model. Using the above path length equations, the modulation ratio N can be expressed as follows:

N₁₂=AC/DC(in-vivo,λ₁)/AC/DC(in-vivo,λ₂)=(−cₐ*εₐ¹*Lₐ¹)/(−cₐ*εₐ²*Lₐ²)=(εₐ¹*PLM¹*lₐ¹)/(εₐ²*PLM²*lₐ²), (8)

where the subscripts and superscripts 1 and 2 refer to the two different wavelengths (λ₁, λ₂).

Because lₐ¹=lₐ², the above equation reduces to:

N₁₂(in-vivo)=PLM¹/PLM²×ε¹/ε²=PLM¹/PLM²×N₁₂(within L-B) (9)

A function g is now defined as the relationship between N₁₂(in-vivo) and N₁₂(within L-B): N₁₂(in-vivo)=g(N₁₂(within L-B)). The transformation from the in-vivo measured modulation ratio to the ideal fictitious modulation ratio in the Lambert-Beer model can then be expressed by the inverse function g⁻¹ as follows:

Nₖₗ(within L-B)=g⁻¹(k,l,tissue properties, Nₖₗ(in-vivo)) (10),
where the transformation depends on the background tissue color, on the color density, and on the wavelengths k and l.

The total hemoglobin, THb, is the tissue parameter that essentially determines the color density of the background. The background color is mainly determined by the arterial and venous saturations and relative arterial and venous volume proportions.

Determination of the Transformations g

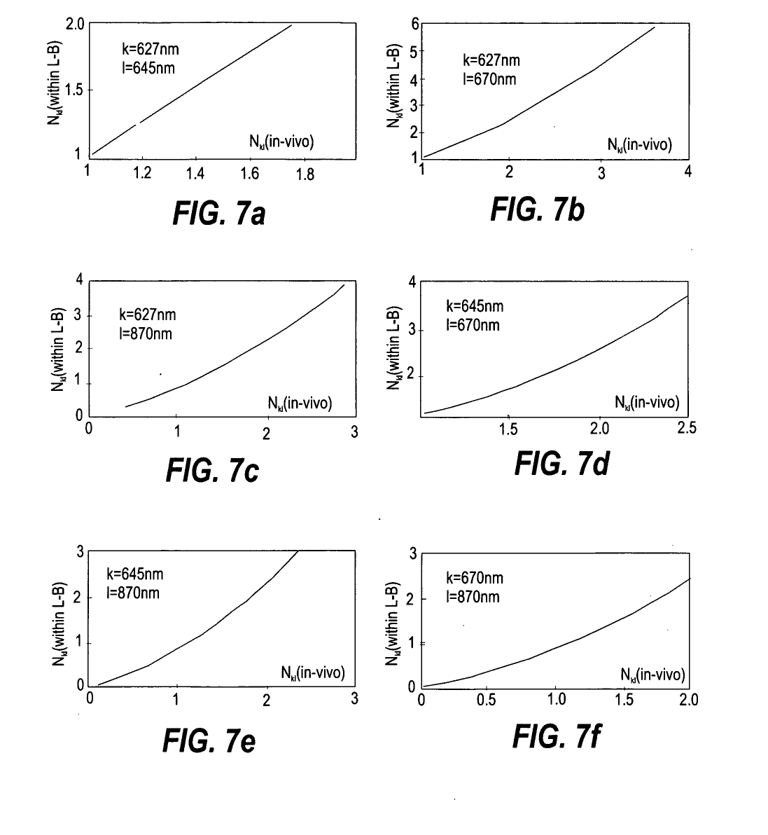

The transformations g(k,l) may be found by the following two methods:
1) Empirically by measuring N(in-vivo) and the concentrations of the different hemoglobin species in blood by a co-oximeter, and then calculating from the hemoglobin concentrations the N(within L-B) for each wavelength pair; or
2) By empirically determining the above relationship for one wavelength pair (optimally 660 nm and 940 nm) and then extrapolating the relationship to other wavelength pairs by using a wavelength dependent tissue model.
   Though the empirical method 1) is possible, it requires a considerable amount of work, because the relationships, such as those in FIGS. 7a to 7f, must be determined for each free tissue parameter, for instance THb, separately. Furthermore, the background tissue properties change the transformations only slightly, and the changes may be masked by the inaccuracies of the measurement itself. Another difficulty is to maintain background properties that are constant enough in a dynamical clinical or laboratory test situation.

Therefore, the above method 2), i.e. the tissue model based approach, is used in this context. Examples of transformations g obtained by this method for nominal a priori tissue parameter values are shown in FIG. 7a to 7f, which illustrate the transformations for the wavelengths of 627, 645, 670, and 870 nm.

The transformations between the Lambert-Beer model and in-vivo measurements are also discussed in U.S. Pat. No. 6,104,938.

The transformations can be presented also as second order polynomies. Table 1 summarizes these polynomies for the above 627-645-670-870 nm pulse oximetry.

TABLE 1
N(within L-B) = a × [N(in-vivo)]² + b × N(in-vivo) + c;

| Wavelengths (nm) | a | b | c |
|---|---|---|---|
| 627, 870 | 0 | 1.323 | −0.320 |
| 645, 870 | 0 | 1.317 | −0.307 |
| 670, 870 | 0.251 | 0.671 | −0.020 |
| 627, 670 | 0.635 | −0.376 | 0.785 |
| 645, 670 | 0.311 | 0.589 | −0.008 |
| 627, 645 | 0.361 | 0.512 | 0.023 |

The Extrapolation of the Standard Oximetry R-Calibration to Other Wavelengths Using an In-Vivo Tissue Model

The transformation g at 660 nm/940 nm, i.e. at the wavelengths of a standard pulse oximeter, is the mapping function from the N-ratio(within L-B) to the N-ratio(in-vivo). This particular transformation can be determined accurately because the empirical relationship is based on thousands of blood samples used to calibrate conventional pulse oximeters operating at the said wavelengths. Therefore, this N-ratio relationship, i.e. function g(660 nm, 940 nm), is first used to establish a realistic tissue model, which will eventually reproduce the calibration for the 660 and 940 nm pulse oximeter. The tissue model is developed with a number of tissue parameters that first assume typical nominal values reflecting the average tissue conditions at the device calibration set-up. One of the model parameters included is the wavelength. Once a satisfactory model with nominal tissue properties is found for the 660/940 nm oximetry, the wavelength dependence is used to extrapolate the in-vivo signals vs. SpO₂ relationships for other wavelength pairs.

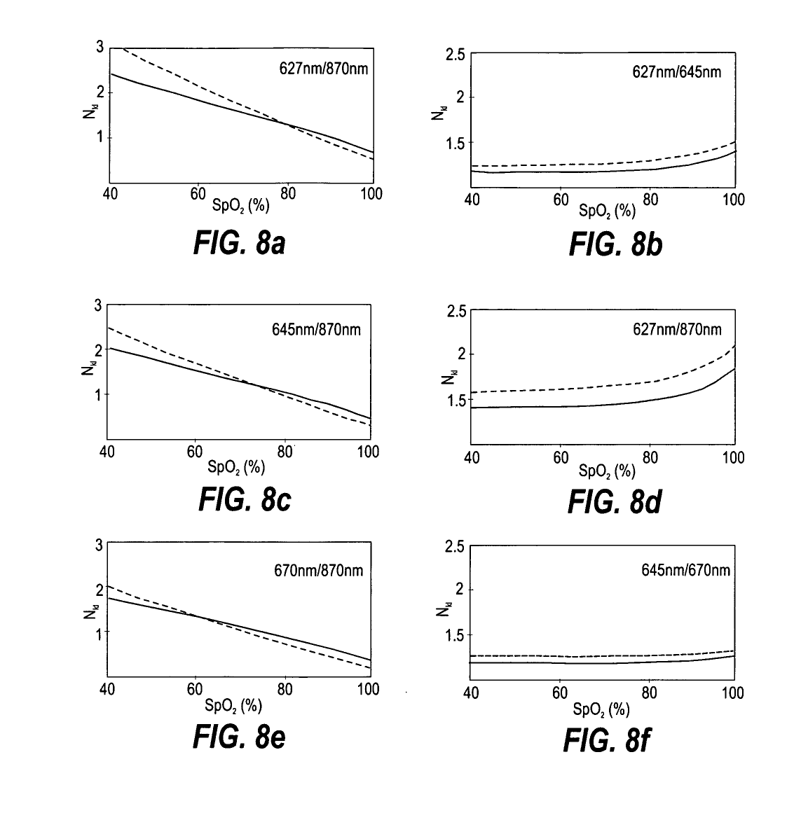

FIG. 8a to 8f represent the in-vivo measured and theoretical Lambert-Beer modulation ratios as a function of SpO₂ for the wavelengths of Table 1. Solid lines represent in-vivo values, while dashed lines represent Lambert-Beer values.

The Parameterized In-Vivo Tissue Model

Using the Monte-Carlo type numeric tissue modeling or other more conventional tissue models, it has been shown that the higher the scattering in the tissue, the longer the actual light path length through the tissue. Furthermore, increased absorption with constant scattering decreases the path length through the tissue. It is therefore reasonable to estimate the actual in-vivo path length using the ratio of the tissue absorption and scattering efficiencies as a parameter in a series expansion of the path length. The series expansion, such as Taylor series expansion, may be derived relative to a very highly scattering medium, i.e. relative to predictions of the diffusion approximation with Σₐ/Σₛ=0. As a result, the path length multiplier PLM may be expressed as follows:

$$PLM = D - B \times ( D - 1 ) \times ( {\sum }_{a} / {\sum }_{s}^{′} ) + ( A / 2 ) \times B \times ( B - 1 ) \times ( D - 1 ) ) \times {( {\sum }_{a} / {\sum }_{s}^{′} )}^{2} \qquad ( 11 )$$

where A, B, and D are series expansion coefficients and

$$\frac{{\sum }_{a} }{{\sum }_{s}^{′} } ( λ ) = \frac{{\sum }_{a} }{{\sum }_{s}^{′} } ( {λ}_{0} ) \times {( \frac{λ}{{λ}_{0}} )}^{N} \times \frac{{\sum }_{a} ( λ )}{{\sum }_{a} ( {λ}_{0} )} = ( ( 1 - bvf ) \times {C}_{tissue} + bvf \times {C}_{blood} \times \frac{THb}{{THb}_{N}} \times \frac{( 1 - {H}_{N} ) \times ( 1.4 - {H}_{N} )}{( 1 - H ) \times ( 1.4 - H )} ) \times {( \frac{λ}{{λ}_{0}} )}^{n} \times \frac{\begin{matrix}bvf \times ( {f}_{a} \times {μ}_{a} ( λ ) + {f}_{v} \times {μ}_{v} ( λ ) ) + \\ wf \times {μ}_{w} ( λ )\end{matrix}}{\begin{matrix}bvf \times ( {f}_{a} \times {μ}_{a} ( {λ}_{0} ) + {f}_{v} \times {μ}_{v} ( {λ}_{0} ) ) + \\ wf \times {μ}_{w} ( {λ}_{0} )\end{matrix}} \qquad ( 12 )$$

where bvf is the blood volume fraction; wf is the water volume fraction; μₐ, μᵥ, and μ_{w} are, respectively, the arterial, venous and water linear absorption coefficients; THb and H refer respectively to total hemoglobin and hematocrit; fₐ and fᵥ are the arterial and venous blood volume fractions; λ is the wavelength; λ₀ is the isobestic (805 nm) wavelength for oxy- and reduced hemoglobin; and the subscript N refers to the nominal value of the respective parameter.

The tissue parameters with their nominal values are summarized in Table 2 below.

TABLE 2

| Model | | Empirical | Nominal |
|---|---|---|---|
| parameters | | Range | value |
| | D | >1 | 3.2 |
| | B | NA | 30 |
| | A | <=1 | 0.75 |
| Tissue | Σₐ/Σ′ₛ for | 0.01-0.02 | 0.02 |
| parameters | bloodless | | |
| | tissue (Cₜᵢₛₛᵤₑ) | | |
| | Wf | 0.6-0.9 | 0.75 |
| | Bvf | 0.01-0.1 | 0.025 |
| | Exponent for | 0.4-2 | 0.4 (900 nm); |
| | wavelength | | 0.9 (660 nm) |
| | dependent | | |
| | scattering [N] | | |
| Blood | THb/THb₀ | 0.5-1.5 | 1 |
| parameters | | | |
| | H | 0.25-0.5 | 0.45 |
| | Σₐ/Σ′ₛ for | 0.1-0.2 | 0.2 |
| | whole blood at | | |
| | 805 nm (C_{blood}) | | |
| | DysHb | 0.01-0.03 | 0.015 |
| | Fa | 0.2-0.4 | 0.25 |
| | Difference of | 5-30% | 10% |
| | the venous and | | |
| | arterial | | |
| | saturation | | |
| | (Vena- | | |
| | Artdiff) | | |

The expression for the term (Σₐ/Σ′ₛ) (Eq. 12), termed expansion parameter in this context, is here determined by utilizing Lambert-Beer compartment model for Σₐ and taking the tissue parameter values from the empirical tissue data available in literature (Table 2).

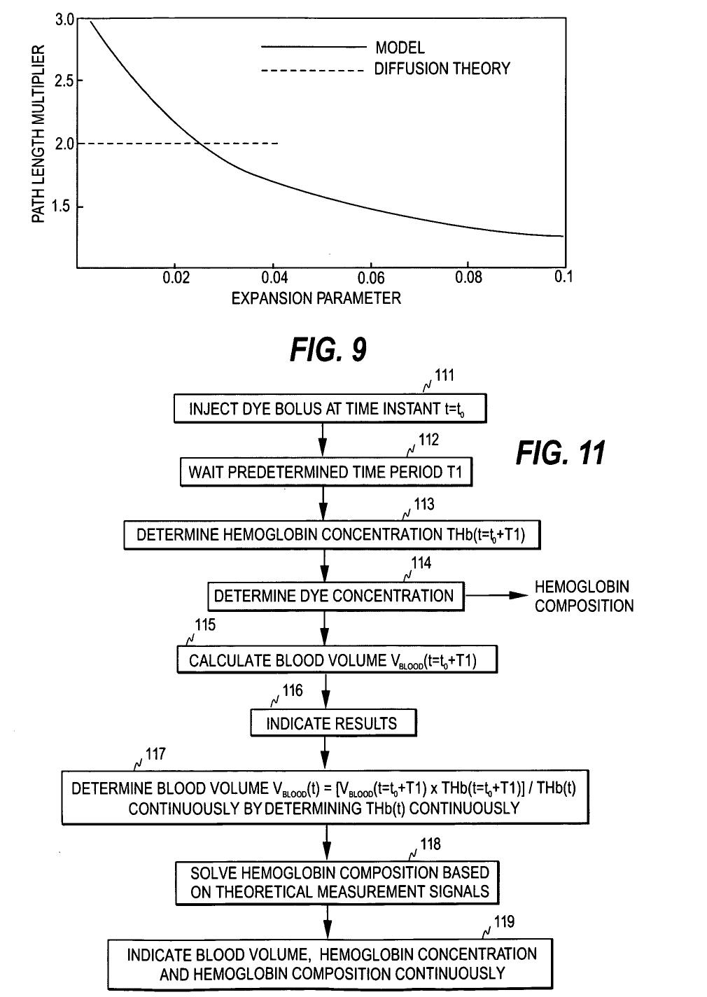

Next, the series expansion coefficients A, B, and D are determined by fitting them so that they reproduce the transformation function g for the conventional 660/940 nm pulse oximeter. The PLM so obtained is presented as a function of the expansion parameter (Σₐ/Σ′ₛ) in FIG. 9. Once the expansion coefficients A, B and D are known, the ratio of the two PLM's (Eq. 11, 12) is calculated at any two desired wavelengths. This ratio determines the transformations g(k,l) (Eq. 10), hereafter termed FRactional OXimetry (FROX) transformation.

Determination of Hemoglobin Using the FROX Transformation and SpO₂

For given transformations g(k,l), the SpO₂ can be obtained from the in-vivo measured N ratios using the equation:

$${SpO}_{2} ( k , l ) = \frac{{ɛ}_{kHb} - {g}_{kl}^{- 1} ( THb ) \times {N}_{kl}^{i n - vivo} \times {ɛ}_{iHb}}{\begin{matrix}{g}_{kl}^{- 1} ( THb ) \times {N}_{kl}^{i n - vivo} \times \\ ( {ɛ}_{{lHbO}_{2}} - {ɛ}_{lHb} ) - ( {ɛ}_{kHbO 2} - {ɛ}_{kHb} )\end{matrix}} , \qquad ( 13 )$$

where N(in-vivo) is denoted with N^{in-vivo}. This equation represents the a priori relationship between SpO₂ and in-vivo measurement signals, cf. step 51. As discussed below, the equation may be stored either in the sensor or in the processing unit of the pulse oximeter.

If the blood contains only two hemoglobin species, oxyhemoglobin (HbO2) and reduced hemoglobin (Hb), the SpO₂ calculated at any two wavelengths must result in the same value. The measurement of hemoglobin can now be based on the PLM model (Eq. 9-12) in which the hemoglobin concentration, THb and H, is adjusted so that all SpO₂(k,l) values calculated according to Equation (13) are essentially the same.

Hemoglobin measurement requires a minimum of three wavelengths (1, 2, 3). SpO₂ may, in this case, be calculated in two independent ways: from N₁₂ and N₁₃. N₂₃ may be calculated from these two as the ratio N₁₃/N₁₂. Therefore, SpO₂(2,3) is not independent as it may be derived from SpO₂(1,2) and SpO₂(1,3). The use of three wavelengths thus allows the determination of the SpO₂ value and one dominating tissue parameter, i.e. THb. The accuracy of THb may be improved by using more wavelengths: with four wavelengths, three independent SpO₂ values may be calculated. This results in an estimate of a true SpO₂ and two free tissue parameters, such as THb and venous saturation. In one embodiment of the present invention, 6 to 8 wavelengths are used, which allows the determination of all important tissue parameters through, respectively, 5 to 7 independent SpO₂ equations, In general, M−2 tissue parameters may be determined based on M wavelengths. It is assumed here that oxyhemoglobin and deoxyhemoglobin (reduced hemoglobin) are the only color components in blood. In presence of dyshemoglobins, more wavelengths are needed to estimate the tissue parameters. For instance, with both methemoglobin (metHb) and carboxyhemoglobin (HbCO) in blood, a minimum of 5 wavelengths are needed for the determination of THb. In this case, it is required that for each possible combination of 4 wavelengths, the same fractional hemoglobin composition shall be obtained.

Above, the predetermined parameter that is employed to detect consistency is the color of arterial blood, that is SpO₂ or the fractions of a predetermined hemoglobin component. This embodiment of the present invention may be summarized so that in the absence of dyshemoglobins a set of M−1 values of the predetermined parameter, i.e. SpO₂(k,l), may be calculated based on signals at M wavelengths. The tissue model, including THb as a tissue parameter, is adjusted to search for the THb values that renders SpO₂(k,l) values the same.

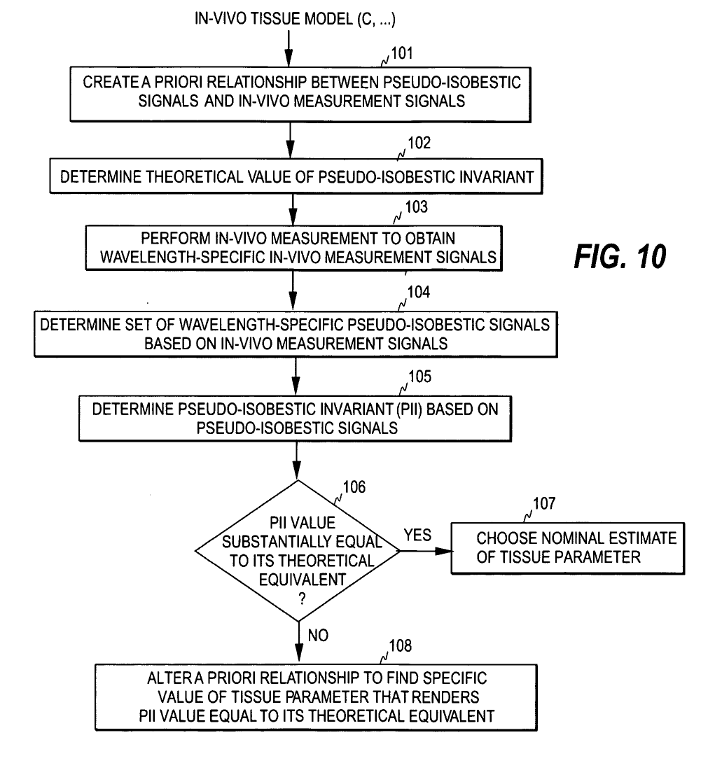

Above, the embodiments utilizing SpO₂ as the predetermined parameter were discussed in more detail. Below, further embodiments are discussed with reference to FIG. 10. The said further embodiment are based on isobestic signals and pseudo-isobestic invariants.

FIG. 10 illustrates an embodiment, in which the predetermined parameter is an isobestic signal. An isobestic signal here refers to a weighted sum of two signals, the weight being selected so that the sum signal is isobestic, i.e. independent of the relative concentrations of the hemoglobin species. In case of an isobestic signal being the parameter reflecting the effect of the tissue on the useful signal, consistency is achieved for the different wavelengths if a quotient of two pseudo-isobestic signals is essentially the same as its theoretical equivalent. The quotient, which is theoretically a constant parameter, is in this context termed pseudo-isobestic invariant (PII). Pseudo-isobestic signals and invariants are discussed in U.S. Pat. No. 6,501,974 B2.

Determination of Hemoglobin Using the FROX Transformation and Pseudo-Isobestic Signals

In this embodiment for determining the hemoglobin concentration, the a priori relationship is thus formed between in-vivo and pseudo-isobestic signals (step 101) and the theoretical value of PII is determined and stored in the apparatus (step 102). Steps 101 and 102 are carried out in the manufacturing phase of the apparatus.

After this, when the apparatus is in use, the in-vivo measurements are made by measuring the transmission signals at three or more wavelengths and at least one in-vivo based value is determined for the PII based on the in-vivo measurement signals and the relationship (step 103-105). The said at least one value is compared with the stored theoretical value of PII (step 106). If the obtained value(s) is/are substantially the same as the theoretical value, the effect of the background on the measurement signal is substantially consistent at the different wavelengths, and the a priori assumption may be regarded as correct (step 107).

However, typically there is a substantial difference between the theoretical value and the in-vivo based value(s) obtained at step 105. The a priori relationship is then altered to find out the concentration value for which the obtained PII value(s) correspond, as accurately as possible, the theoretical value of the PII (step 108).

Below, the embodiment of FIG. 10 is discussed in more detail.

A photon hitting the detector at each wavelength must cross the same thickness or the same number of Hb molecules of pulsating arterial blood in the Lambert-Beer tissue model. In other words, cₐ×lₐ, i.e. the product of the hemoglobin concentration and blood volume thickness, is an invariant. As can be seen from equation (1), the Lambert-Beer modulation ratio AC/DC(within L-B) is proportional to this invariant, the proportionality coefficient being the extinction coefficient of pulsating arterial blood. Next, a new set of equations is constructed so that other estimates for the tissue model parameters may be determined.

Pseudo-Isobestic Invariant, PII

‘The color’ is first eliminated from the signals. The color is an invariant in the SpO₂ parameter; optimally the new set of parameters can best complement the color invariant, if the color is eliminated from the set of new equations. At the isobestic point of oxyhemoglobin and deoxyhemoglobin the signal does not depend on the relative proportions of the hemoglobin fractions, i.e. the signal is color invariant. A color invariant signal may be calculated from two color dependent signals by summing the signals in the proportions that lead to invariancy. The resulting signal (within L-B), which is called pseudo-isobestic signal, may then be defined in the following way:

S(k,l)=dAₖ+f_{k,l}×dAₗ=dA×(εₖ^{RHb}+f_{k,l}×εₗ^{RHb}) (14)
where

$${f}_{k , l} = \frac{{ɛ}_{k}^{RHb} - {ɛ}_{k}^{hbO 2}}{{ɛ}_{l}^{RHb} - {ɛ}_{l}^{HbO 2}}$$

and dA is a common factor proportional to cₐ*lₐ, i.e. to the number of hemoglobin molecules in the pulsating arterial blood.

The pseudo isobestic invariant within Lambert-Beer is the ratio of two pseudo-isobestic signals. This ratio is independent of both the color, the volume, and the THb of blood. The pseudo isobestic invariant PII may be written in the following way:

$$PII ( k , l , m , n ) = \frac{dA \times ( {ɛ}_{k}^{RHb} + {f}_{k , l} \times {ɛ}_{l}^{RHb} )}{dA \times ( {ɛ}_{m}^{RHb} + {f}_{m , n} \times {ɛ}_{n}^{RHb} )} \qquad ( 15 )$$

Using the actual in-vivo measured signals, PII can be written as follows:

$$PII ( k , l , m , n ) = \frac{{dA}_{k} + {f}_{k , l} \times {dA}_{l}}{{dA}_{m} + {f}_{m , n} \times {dA}_{n}} = \frac{{dA}_{l}}{{dA}_{n}} \times \frac{\frac{{dA}_{k}}{{dA}_{l}} + {f}_{k , l}}{\frac{{dA}_{m}}{{dA}_{n}} + {f}_{m , n}} = {N}_{l n}^{L - B} \times \frac{{N}_{kl}^{L - B} + {f}_{k , l}}{{N}_{mn}^{L - B} + {f}_{m , n}} = {g}_{l n}^{- 1} ( {N}_{l n}^{i n - vivo} ) \times \frac{{g}_{kl}^{- 1} ( {N}_{kl}^{i n - vivo} ) + {f}_{k , l}}{{g}_{mn}^{- 1} ( {N}_{mn}^{i n - vivo} ) + {f}_{m , n}}$$

where N(in-vivo) and N(within L-B) are denoted respectively with N^{in-vivo} and N^{L-B}. At least three wavelengths are needed to calculate a PII from the measured signals. For 4 wavelengths 2 independent PIIs (total 15 PII's) can be calculated: For M wavelengths M−2 independent PIIs can be calculated.

Each PII within Lambert-Beer is a constant, and in principle independent of blood color (SpO₂) or color density (blood volume and THb). However, if the transformation g is not correct in Eq. 10, the value of PII determined based on the measured signals may differ from its theoretical constant value. The total hemoglobin THb and other parameters in the tissue model can now be adjusted so that the predetermined theoretical PII values are obtained. The tissue model parameters that render the PII invariant determine the total hemoglobin THb in blood.

The above two embodiments, in which the predetermined parameter is, respectively, SpO₂ or PII, may also be combined so that both SpO₂ and PII are employed. This method may be summarized as follows:

An in-vivo tissue model is first constructed. Within this model an expression for the path length multiplier is defined at each wavelength.
6-8 wavelengths are selected and pulse oximeter measurement is performed at all wavelengths.
The transformations g from the measured in-vivo N-ratio to the theoretical Lambert-Beer N-ratio is calculated for each wavelength pair. Nominal tissue parameter values are used for the nominal g functions.
A first predetermined parameter, SpO₂, is calculated using the nominal transformations. M−1 different and independent SpO₂ values can be determined for M different wavelengths.
A second predetermined parameter, PII, is calculated using all wavelength signals. N−2 different and independent PIIs can be calculated.
The functions g are altered by altering the tissue model parameters, including THb and H, until:
   1. The calculated SpO₂ values are all the same or almost the same,
   2. the calculated PIIs match or almost match with their theoretical constant values.
Finally, the THb and H, which produce the closest agreement with the measured signals and the predetermined invariants, are the desired hemoglobin concentration and hematocrit values.

Above, the determination of hemoglobin concentration, cf. steps 21-22 in FIG. 2 and steps 30-32 in FIG. 3, was discussed in detail. As also discussed in connection with FIG. 3, blood volume and possibly also hemoglobin composition may be determined, in addition to hemoglobin concentration, in the continuous measurement state of the apparatus. This is discussed in the following.

Determination of the Blood Volume from Hemoglobin Concentration THb

As can be seen from the hemodilution equation discussed in connection with FIG. 2, the determination of blood volume requires the determination of the term V_{blood}(t)×THb(t) at time instant t=t₀. In a situation where the patient is leaking or when the blood volume changes rapidly, for example when a rapid volume expansion is needed for stabilizing the patient hemodynamics, there is usually no time to measure V_{blood}(t₀). However, as a simple way to measure the value of V_{blood}(t₀) is disclosed here, the said measurement may be carried out anytime when the situation is convenient, for instance regularly in the beginning of a risk surgical procedure. This is discussed below in connection with FIG. 11.

A dye dilution can be employed in a novel way within the above hemoglobin concentration calculation algorithm. This procedure is described below for a pulse oximeter provided with three wavelengths with three unknown concentrations: HbO2, RHb, and dye substance. Any two wavelengths are used for determining the SpO₂ value and any remaining wavelength to simultaneously determine the concentration of the intra-venous dye. A three wavelength oximeter is selected here for its simplicity, but a better accuracy may be achieved with a 6 or 8 wavelength oximetry platform.

Measurement of the Absolute Blood Volume Using Intra-Venous Dyes in a Three Wavelength Oximetry

In a two wavelength oximetry, using the FROX oximetry principles the SpO₂ may be solved in the Lambert-Beer ideal cuvette model using the ideal Lambert-Beer extinction coefficients by solving a set of linear equations:

$$( \begin{matrix}{dA}_{1} \\ {dA}_{2}\end{matrix} ) = ( \begin{matrix}{ɛ}_{1}^{HbO 2} & {ɛ}_{1}^{RHb} \\ {ɛ}_{2}^{HbO 2} & {ɛ}_{2}^{RHb}\end{matrix} ) * ( \begin{matrix}{SpO}_{2} \\ 1 - {SpO}_{2}\end{matrix} ) .$$

The signals dAᵢ (i=1, 2) are the pathlength-transformed in-vivo signals describing the ideal non-scatter signals, i.e., theoretical measurement signals.

As discussed above, N₁₂(within L-B)=dA₁/dA₂=g⁻¹ (N₁₂(in-vivo)).

This leads to Eq. (3), when SpO₂ is solved from the matrix equation. In case of OxyHb and RHb are the only hemoglobins in blood and a bolus of intra-venous dye is injected into the subject's blood circulation, the hemoglobin and dye concentrations may be solved, in a three wavelength oximetry, from the following equation:

$$( \begin{matrix}{dA}_{1} \\ {dA}_{2} \\ {dA}_{3}\end{matrix} ) = ( \begin{matrix}{ɛ}_{1}^{HbO 2} & {ɛ}_{1}^{RHb} & {ɛ}_{1}^{D} \\ {ɛ}_{2}^{HbO 2} & {ɛ}_{2}^{RHb} & {ɛ}_{2}^{D} \\ {ɛ}_{1}^{HbO 2} & {ɛ}_{3}^{RHb} & {ɛ}_{3}^{D}\end{matrix} ) * ( \begin{matrix}{SpO}_{2} \\ 1 - {SpO}_{2} \\ D\end{matrix} ) , \qquad ( 16 )$$

where εᵢ^{X} is the ideal non-scatter extinction coefficient for the hemoglobin derivatives (X=HbO2, RHb) and for the intravenous dye concentration (X=D), where D is normalized with respect to the total hemoglobin concentration (=1), and dAᵢ, i=1, 2, 3 are the path length transformed in-vivo signals at wavelengths 1, 2, and 3, respectively.

It is important to note that the transformations depend only on the total absorption and scattering efficiencies and not on the particular hemoglobin composition. Therefore, THb can still be determined as described above (as it is the variable in the transforming function g). However, a fourth wavelength is needed for simultaneous measuring both THb and D. Furthermore, hemoglobin composition may be determined based on Eq. (16) even after the dye substance has been eliminated from the body, i.e., when Eq. (16) yields D=0.

The steps for determining the blood volume are now discussed with reference to FIG. 11. It is assumed here that the hemoglobin concentration is determined substantially continuously in the manner described above.

An intravenous dye bolus containing a predetermined concentration of the dye, such as Indocyanine Green, is prepared and injected to the subject at time instant t=t₀ (step 111). It is further assumed here that the volume and concentration of the bolus are B dl and C g/dl, respectively. A predetermined time period T1 is then waited to ensure that the bolus is evenly distributed within the blood circulation of the subject before the process continues (step 112). After the said period, hemoglobin concentration is determined in step 113 in the above-discussed manner by adjusting the transformations so that the same SpO₂ values are obtained consistently for all wavelength combinations (step 57 in FIG. 5) or so that the PII value is substantially equal to its theoretical equivalent (step 108 in FIG. 10). Furthermore, dye concentration D is determined in step 114 by solving the linear set of Eq. (16) modified for the number of wavelengths in use. After this, the hemoglobin composition is known. The blood volume may now be calculated as follows based on D: V_{blood} (t+T1)×THb(t+T1)=(C/D)×B (step 115). The hemoglobin concentration and composition and the blood volume obtained at t to may now be indicated to the end-user (step 116). Once the blood volume at t=t₀+T1 is known, the blood volume may be determined continuously based on the above equation

$${V}_{blood} ( t ) = {V}_{blood} ( {t}_{0} + T 1 ) \times \frac{THb ( {t}_{0} + T 1 )}{THb ( t )} = ( \frac{C}{THb ( t )} ) \times ( \frac{B}{D ( {t}_{0} + T 1 )} )$$

continuously determining THb(t) (step 117). The determination of hemoglobin composition may be combined with the determination of hemoglobin concentration in the continuous state (step 118). In other words, simultaneously as the THb(t) is determined, hemoglobin composition may be determined based on Eq. (16). The apparatus may thus continuously indicate the blood volume, hemoglobin concentration, and hemoglobin composition of the subject (step 119). Steps 117-119 therefore represent the continuous state of the measurement. Steps 111-116 in turn correspond to the initial volume calibration which may be carried out anytime when the situation is convenient for the injection of the dye bolus, such as before a risk surgical procedure. The initial volume calibration may also be performed in good time before such a procedure, since the continuous state of the measurement may be maintained for longer periods without any harm to the patient.

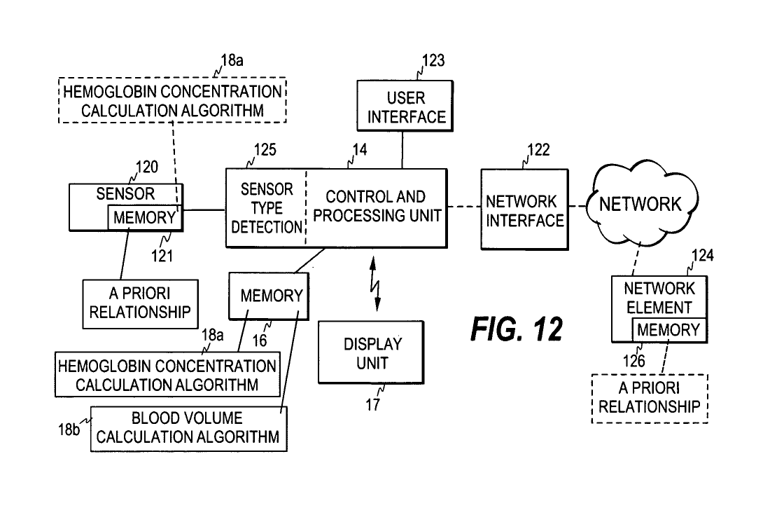

The pulse oximeter of FIG. 1 includes the algorithms 18a and 18b configured to carry out the above steps. Furthermore, the a priori relationship for the determination of the hemoglobin concentration is stored in the memory of the pulse oximeter in the manufacturing phase of the apparatus. However, it is to be noted that that all the operations are not necessarily carried out in the actual pulse oximeter or in its control and processing unit, but the entities carrying out the operations may be distributed between a sensor attached to the patient, a central unit, and/or a communication network. For example, the a priori relationship may be stored in any of these locations. Furthermore, the elements that determine the hemoglobin concentration, hemoglobin composition and blood volume may reside in the central unit or be distributed between two or more of these possible locations or within the network. For example, the storing of the a priori relationship and the determination of the above parameters may take place in various processing units of a network, such as the local area network of a hospital. FIG. 12 illustrates an example of an apparatus in which the a priori relationship is stored in the memory 121 of a sensor 120 attachable to the patient, whereas the data processing entities are in the central unit 14 of the pulse oximeter. Furthermore, in this example, the connection between the central unit 14 and the monitor 17 is wireless. Any appropriate short-range wireless radio technology may be used to transfer the data from the central unit to the monitor. The pulse oximeter may also be provided with a network interface 122 for downloading/updating the a priori relationship through a network from a network element 124 storing the a priori relationship. This is illustrated with dotted lines in the figure.

The sensor may also be provided with data processing capability and may be connected to the central unit directly or through the local area network, for example. The connection from the sensor to the central unit or to the local area network may be a wired or wireless connection. In one embodiment, the sensor attachable to the subject may be configured to determine the hemoglobin concentration. In this case the sensor may store the necessary information for the determination of the hemoglobin, such as the a priori relationship and the hemoglobin concentration calculation algorithm, and the necessary parameters, such as the extinction coefficients. The sensor may send the hemoglobin concentration values to the central unit through a hospital LAN, for example, and the central unit may determine the blood volume and/or its trend and display the hemoglobin concentration/composition and the blood volume status (absolute volume and/or volume trend) to the end-user. For example, the central unit may in this case be a ward server that may locate in a ward control room.

In one embodiment, the central unit is compatible with both a conventional sensor (two wavelengths) and an advanced sensor capable of monitoring blood volume status (three or more wavelengths and optional data, depending on how the blood volume is monitored and which of the above parameters are determined). The central unit may be provided with a recognition module 125 for recognizing the type of the sensor. If the recognition module detects that an advanced sensor capable of monitoring the blood volume status is connected to it, it may download data from the sensor and/or network according to the parameters to be determined and displayed. Such an advanced oximeter may display the hemoglobin concentration, the hemoglobin composition, and the blood volume substantially continuously, as is discussed in connection with FIG. 11. The user of the device may configure the parameters to be displayed through a user interface 123.

A pulse oximeter may also be upgraded to a device capable of monitoring the blood volume status of a patient. Such an upgrade may be implemented by delivering to the pulse oximeter a software module that enables the device to carry out the above steps. The software module may be delivered, for example, on a data carrier, such as a CD or a memory card, or through a telecommunications network. The software module may store the a priori relationship or may be provided with access to an external memory holding the a priori relationship, for example.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for monitoring blood volume status of a subject, the method comprising:
acquiring in-vivo measurement signals at a plurality of measurement wavelengths, the in-vivo measurement signals being indicative of absorption caused by blood of a subject;
determining, based on the in-vivo measurement signals, a hemoglobin measure indicative of concentration of hemoglobin in the blood of the subject, wherein the determining includes determining successive values of the hemoglobin measure; and
monitoring, based on the successive values of the hemoglobin measure, the blood volume of the subject.

2. The method according to claim 1, wherein the determining includes
creating an a priori relationship indicative of effect of tissue on the in-vivo measurement signals; and
determining, based on the a priori relationship, a specific value of the hemoglobin measure for which the effect of the in-vivo tissue on in-vivo measurement signals is consistent for the plurality of measurement wavelengths, the specific value representing a value of the hemoglobin measure, wherein the value is any of the successive values.

3. The method according to claim 2, wherein the creating includes creating the a priori relationship through an in-vivo tissue model including a nominal estimate of the hemoglobin measure.

4. The method according to claim 1, wherein the monitoring includes estimating a value for the blood volume of the subject.

5. The method according to claim 4, wherein the estimating includes
injecting dye substance into the blood of the subject;
determining concentration of the dye substance in the blood of the subject; and
defining the value for the blood volume based on the concentration of the dye substance.

6. The method according to claim 5, wherein the monitoring further includes determining changes in the hemoglobin measure relative to a given time instant at which the value for the blood volume is defined based on the concentration of the dye substance.

7. The method according to claim 1, further comprising determining hemoglobin composition of the subject.

8. The method according to claim 1, wherein the monitoring includes monitoring relative changes in the blood volume of the subject.

9. An arrangement for monitoring blood volume status of a subject, the arrangement comprising:
a signal reception unit configured to receive in-vivo measurement signals corresponding to a plurality of measurement wavelengths, the in-vivo measurement signals being indicative of absorption caused by blood of a subject;
a hemoglobin determination unit configured to determine, based on the in-vivo measurement signals, a hemoglobin measure indicative of concentration of hemoglobin in the blood of the subject, wherein the hemoglobin determination unit is configured to determine successive values of the hemoglobin measure; and
a monitoring unit configured to monitor, based on the successive values of the hemoglobin measure, the blood volume of the subject.

10. The arrangement according to claim 9, further comprising a sensor attachable to the subject, the sensor comprising:
an emitter unit configured to emit radiation through tissue of the subject at the plurality of measurement wavelengths;
a detector unit configured receive the radiation and to produce the in-vivo measurement signals corresponding to the plurality of wavelengths.

11. The arrangement according to claim 9, wherein the hemoglobin determination unit is configured to
retrieve an a priori relationship indicative of effect of tissue on in-vivo measurement signals at the plurality of measurement wavelengths; and
determine, based on the a priori relationship, a specific value of the hemoglobin measure for which the effect of the in-vivo tissue on in-vivo measurement signals is consistent for the plurality of measurement wavelengths, the specific value representing a value of the hemoglobin measure, wherein the value is any of the successive values.

12. The arrangement according to claim 11, wherein the a priori relationship is created through an in-vivo tissue model including a nominal estimate of the hemoglobin measure.

13. The arrangement according to claim 9, wherein the monitoring unit is configured to estimate a value for the blood volume of the subject.

14. The arrangement according to claim 13, wherein the monitoring unit is configured to
determine concentration of a dye substance in blood of the subject; and
define the value for the blood volume based on the concentration of the dye substance.

15. The arrangement according to claim 14, wherein the monitoring unit is further configured to determine changes in the hemoglobin measure relative to a given time instant at which the value for the blood volume is defined based on the concentration of the dye substance.

16. The arrangement according to claim 13, wherein the monitoring unit is further configured to determine hemoglobin composition of the subject and the monitoring unit comprises a display for displaying the hemoglobin concentration, hemoglobin composition, and blood volume.

17. The arrangement according to claim 9, wherein the monitoring unit is configured to monitor relative changes in the blood volume of the subject.

18. A sensor for determining blood volume status of a subject, the sensor being attachable to the subject and comprising:
an emitter unit configured to emit radiation through the tissue of the subject at a plurality of measurement wavelengths;
a detector unit configured to receive the radiation and to produce in-vivo measurement signals corresponding to the plurality of measurement wavelengths, wherein the in-vivo measurement signals are indicative of absorption caused by blood of the subject;
a hemoglobin determination unit configured to determine, based on the in-vivo measurement signals, a hemoglobin measure indicative of concentration of hemoglobin in the blood of the subject, wherein the hemoglobin determination unit is configured to determine successive values of the hemoglobin measure;
an interface unit configured to send the successive values of the hemoglobin measure to an external unit configured to monitor the blood volume of the subject based on the successive values.

19. An apparatus for monitoring blood volume status of a subject, the apparatus comprising:
a reception unit configured to receive successive values of a hemoglobin measure indicative of hemoglobin concentration in blood of a subject; and
a monitoring unit configured to monitor, based on the successive values of the hemoglobin measure, the blood volume of the subject.

20. A computer program product for monitoring blood volume status of a subject, the computer program product comprising:
a first program product portion configured to receive in-vivo measurement signals corresponding to a plurality of measurement wavelengths, the in-vivo measurement signals being indicative of absorption caused by blood of a subject;
a second program product portion configured to determine, based on the in-vivo measurement signals, successive values of a hemoglobin measure indicative of hemoglobin concentration in the blood of the subject; and
a third program product portion configured to indicate, based on the successive values, blood volume status of the subject.
